(19) United States
(12) Patent Application Publication
(10) Pub. No.: US 2026/0114954 A1
(43) Pub. Date: Apr. 30, 2026

(54) INFORMATION INDICATORS FOR MEDICAL CONTAINERS

(71) Applicant: Alexander Christopher Ebner, Palm Harbor, FL (US)

(72) Inventor: Alexander Christopher Ebner, Palm Harbor, FL (US)

(21) Appl. No.: 19/375,073

(22) Filed: Oct. 30, 2025

Related U.S. Application Data
(60) Provisional application No. 63/714,218, filed on Oct. 31, 2024.

(51) Int. Cl. A61B 90/96; A61B 50/33; A61L 2/28

(52) U.S. Cl. CPC A61B 90/96; A61B 50/33; A61L 2/28; A61L 2103/15

(57) ABSTRACT

This disclosure is directed to an information indicator assembly and a medical container comprising the same. The information indicator assembly can include an information plate and a plate holder defining a holder recess configured to receive the information plate and further defining a plurality of holder openings that are spatially aligned to match the plurality of container openings of a first medical container. The information plate can include a machine readable indicium, a human readable indicium, or both. The information indicator assembly can include at least one fastener configured to extend through one of the plurality of holder openings and one of the plurality of container openings to thereby couple the plate holder to the medical container.

### CROSS-REFERENCE TO RELATED APPLICATION

This application claims the benefit of U.S. Provisional Application No. 63/714,218, filed Oct. 31, 2024, the disclosure of which is incorporated by reference herein in its entirety.

### FIELD

The present disclosure is directed to an information indicators, and more specifically information indicators for medical containers, for example, surgical and sterilization trays.

### BACKGROUND

Medical containers, for example, sterilization trays, are used to hold one or more medical instruments or devices for storage or during a medical procedure. Medical containers can use medical tape as a label substrate that provides information about the medical container or its contents.

However, the Joint Commission, Association for the Advancement of Medical Instrumentation (AAMI), industry journals, and the FDA have called for the elimination of medical tape in medical container identification and sterilization processes. Medical tape applied to medical containers holding one or more medical instruments traditionally needs frequent replacement and leaves bioburden residue on the sterilization trays and the instruments in the medical containers, which presents risks for cross-contamination and transmission of blood-borne pathogens.

Accordingly, there is a need for alternatives to tape-type products for medical containers that can improve efficiencies, streamlining the medical container identification and sterilization processes with the data provided, scanned, or read from the medical containers.

### BRIEF SUMMARY

In some embodiments, an information indicator assembly for medical containers defining a plurality of container openings. The information indicator assembly can include an information plate having a human readable indicium, a machine readable indicium, or both. The information indicator assembly can also include a plate holder defining a holder recess configured to receive the information plate. The plate holder can define a plurality of holder openings that are spatially aligned to match the plurality of container openings of a first medical container. The information indicator assembly can also include at least one fastener configured to extend through one of the plurality of holder openings and one of the plurality of container openings to thereby couple the plate holder to the medical container.

In some embodiments, a medical container for holding one or more medical instruments can include a body defining an instrument cavity for containing the one or more medical instruments. The medical container can also include an information plate having a human readable indicium, a machine readable indicium, or both; and a plate holder. The plate holder can include a wall surface, a first tab extending from a first side of the wall surface, a second tab extending from a second side of the wall surface, and a third tab extending from a third side of the wall surface opposite the second side. The wall surface, the first tab, the second tab, and the third tab collectively define a holder recess configured to receive the information plate. The medical container can also include a retention mechanism in the holder recess configured to couple the information plate to the plate holder.

### BRIEF DESCRIPTION OF THE DRAWINGS

The accompanying figures, which are incorporated herein, form part of the specification and illustrate embodiments of this disclosure. Together with the description, the figures further serve to explain the principles of and to enable a person skilled in the relevant arts to make and use the various embodiments.

FIG. 1 depicts a front perspective view of an exemplary medical container with an information indicator assembly according to some embodiments.

FIG. 2 depicts a front view of the exemplary information indicator assembly of FIG. 1, according to some embodiments.

FIG. 3 depicts another front perspective view of the exemplary information indicator assembly of FIG. 2, according to some embodiments.

FIG. 4 depicts a top view of the exemplary information indicator assembly of FIG. 2, according to some embodiments.

FIG. 5 depicts a bottom view of the exemplary information indicator assembly of FIG. 2, according to some embodiments.

FIG. 6 depicts a right side view of the exemplary information indicator assembly of FIG. 2, according to some embodiments. The left side view (not shown) of the exemplary information indicator assembly of FIG. 2 can be a mirror image of the right side view.

FIG. 7 depicts a rear view of the exemplary information indicator assembly of FIG. 2, according to some embodiments.

FIG. 8 depicts a front view of an exemplary information plate according to some embodiments.

FIG. 9 depicts a right side view of the exemplary information plate of FIG. 8, according to some embodiments. The left side view (not shown) of the exemplary information indicator assembly of FIG. 2 can be the same as the right side view.

### DETAILED DESCRIPTION

Provided herein are system, apparatus, device, and/or method aspects, and/or combinations and sub-combinations thereof, for an information indicator assembly.

Embodiments will now be described in detail with reference to embodiments thereof as illustrated in the accompanying drawings. References to “one embodiment,” “an embodiment,” “an example embodiment,” “some embodiments,” “exemplary,” etc., indicate that the embodiment described can include a particular feature, structure, or characteristic, but every embodiment may not necessarily include the particular feature, structure, or characteristic. Further, when a particular feature, structure, or characteristic is described in connection with an embodiment, it is submitted that it is within the knowledge of one skilled in the art to affect such feature, structure, or characteristic in connection with other embodiments whether or not explicitly described.

Unless clearly indicated to the contrary (e.g., “either x or y, but not both x and y”) or readily contextually apparent, the term “or” as used herein is inclusive (i.e., “x or y” includes just x, just y, and x and y, and “x, y, or z” includes just x, just y, just z, and any combination thereof). Moreover, such phrases are not necessarily referring to the same embodiment.

The term “about” or “substantially” or “approximately” as used herein means the value of a given quantity that can vary based on a particular technology. Based on the particular technology, the term “about” or “substantially” or “approximately” can indicate a value of a given quantity that varies within, for example, 0.1-10% of the value (e.g., ±0.1%, ±1%, ±2%, ±5%, or ±10% of the value).

Numerical values, including endpoints of ranges, can be expressed herein as approximations preceded by the term “about,” “substantially,” “approximately,” or the like. In such cases, other embodiments include the particular numerical values. Regardless of whether a numerical value is expressed as an approximation, two embodiments are included in this disclosure: one expressed as an approximation, and another not expressed as an approximation. It will be further understood that an endpoint of each range is significant both in relation to another endpoint, and independently of another endpoint. Additionally, it will be further understood that a range expressed as “between” “X” and “Y” includes any value interposed by X and Y as well as X and Y.

The following examples are illustrative, but not limiting, of the present embodiments, other suitable modifications and adaptations of the variety of conditions and parameters normally encountered in the field, and which would be apparent to those skilled in the art, are within the spirit and scope of the disclosure.

Embodiments of this disclosure include an information indicator assembly for medical containers (for example, trays such as surgical, sieve, or sterilization trays, sterilization containers, or sterile wrap) having an information plate and a plate holder. The information plate can include at least a machine readable indicium, such as a barcode (for example, a datamatrix or QR code) that provides hospitals and healthcare staff the ability to efficiently identify and track the medical container, as well as the medical instrument sets contained within the medical container, in real time. The information plate can provide labeling, identification, and tracking functionality without the need for medical tape, which requires frequent replacement and risks leaving bioburden residue (e.g., blood borne pathogens) on the medical containers.

For example, the information indicator assembly can be used with medical containers in operating rooms, sterile processing departments, etc. Each medical instrument placed on or in the medical container can be scanned and associated with a particular medical container. Accordingly, operating room and material managers can assess redundant and/or unused medical instruments, streamlining purchases of medical instruments based on the best value and current needs of practicing surgeons. Budgets and costs on unnecessary repairs and/or replacements can be assessed, and more informed healthcare purchasing decisions can be made.

The information plate can provide various types of information using human-readable indicia (for example, graphical symbols or alphanumeric text), machine-readable indicia (for example, a barcode or a near-field communication tag), or both. For example, this information can include information that uniquely identifies the medical container, information that identifies the medical instruments contained therein, manufacturer details, sterility status, sterilization method or requirements, materials used and any natural rubber or latex content, usage- and safety-related data, instructions for use, reprocessing guidance, repair/replacement frequency and costs, and financial evaluation for reusable medical containers or instruments, indication whether the container and instruments therein are single-use or reusable items, and product description and intended application or procedures (including hospital or medical provider identification).

The plate holder is configured to securely couple the plate to the medical container. In some embodiments, the plate holder is a separate component from the medical container and coupled to the medical container using, for example, one or more fasteners. In other embodiments, the plate holder is an integral component of the medical container.

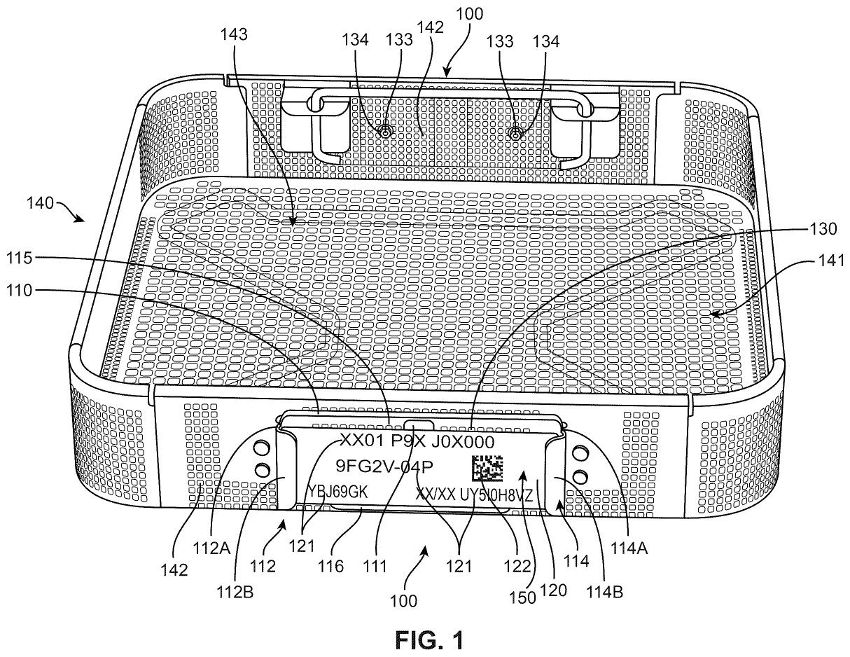

FIGS. 1-9 illustrate an information indicator assembly 100 according to some embodiments. Information indicator assembly 100 includes at least a plate holder 110 and an information plate 120.

Information indicator assembly 100 can be coupled to a medical container 140 (for example, a perforated surgical or sterilization tray as shown in FIG. 1). In some embodiments, one or more information indicator assemblies 100 can be coupled to medical container 140. For example, as shown in FIG. 1, two information indicator assemblies 100 can be coupled to medical container 140—on opposing sides of medical container 140. In some embodiments (not shown), two or more information indicator assemblies 100 can be coupled to one side of medical container 140.

Medical container 140 can be configured to hold one or more medical instruments. In some embodiments, medical container 140 can include a body 141 that defines an instrument cavity 143 for containing one or more medical instruments.

Medical container 140, including body 141, can be composed of a variety of materials (e.g., stainless steel, aluminum, titanium, plastics, Poly Ether Ether Ketone (PEEK), etc.). For example, medical container 140 can be made of stainless steel or aluminum.

In some embodiments, medical container 140 can include a plurality of container openings 142. For example, plurality of container openings 142 can be perforated holes defined in body 141 as shown in FIG. 1. The perforated holes can be configured to allow sufficient drainage during sterilization reprocessing. In some embodiments, plurality of container openings 142 can be circles (as shown in FIG. 1), squares, diamonds, or any other suitable shape.

In some embodiments, plate holder 110 can be a separable component fastened to body 141 of medical container 140. For example, information indicator assembly 100 can also include at least one fastener that couples plate holder 110 to body 141. As shown in FIGS. 1 and 3-7, information indicator assembly 100 can include at least one fastener 132A, 132B. In some embodiments, information plate 120, plate holder 110, and at least one fastener 132A, 132B can be separable components relative to each other. In some embodiments, information plate 120, plate holder 110, and at least one fastener 132A, 132B can be pre-assembled and attached to medical container 140.

In some embodiments (not shown), plate holder 110 can be integral with body 141 of medical container 140. For example, plate holder 110 can be welded to body 141 or otherwise formed to be a unitary structure with medical container 140.

In some embodiments, information plate 120 can be color-coded based on identification or specification needs of a user. For example, information plate 120 can have a color that has a known association with a specific procedure or medical instrument contents within a particular container.

In some embodiments, information plate 120 can be composed of a variety of materials (e.g., stainless steel, aluminum, titanium, plastics, PEEK, etc.). For example, in some embodiments, information plate 120 can be made of anodized aluminum.

In some embodiments, as shown for example in FIGS. 1-3 and 8, information plate 120 can include at least one human-readable indicium 121 and at least one machine-readable indicium 122. In some embodiments (not shown), information plate 120 can include two machine-readable indicium 122. In some embodiments, human-readable indicium 121 and machine-readable indicium 122 are formed on information plate 120 without using medical tape.

For example, in some embodiments, human-readable indicium 121 and machine-readable indicium 122 can be mechanically formed onto information plate 120 by laser etching; dot peen marking; mechanical or chemical engraving; casting, forging, or molding; laser bonding; or mechanical fasteners (e.g., rings, clamps, steel cables, etc.). In other embodiments, human readable indicium 121 and machine readable indicium 122 are formed by ink jet printing or with permanent adhesive labels.

In some embodiments, human-readable indicium 121 or machine readable indicium 122 can include information that uniquely identifies medical container 140. In some embodiments, the information that identifies medical container 140 can include a unique device identifier (UDI). In some embodiments, the UDI can identify one or more medical instruments contained in medical container 140—there is a known association between the UDI and the medical instrument(s) contained in the container. In some embodiments, human-readable indicium 121 or machine readable indicium 122 can include information that indicates the sterilization status, working conditions, and/or surgical readiness of the medical container or the one or more medical instruments contained in the medical container 140. For example, the sterilization status, working conditions, and/or surgical readiness can be associated with the information that identifies medical container 140, e.g., a UDI.

In some embodiments, human-readable indicium 121 can identify a medical facility (e.g., hospital name, hospital address, surgery division/department, etc.). In some embodiments, human-readable indicium 121 can identify a medical provider (e.g., surgeon name).

In some embodiments, machine readable indicium 122 is optically scannable, for example, by using a camera or scanner. Scannable machine readable indicium 122 can provide sterile processing professionals and surgical teams with actionable confirmation of medical instrument readiness in real time when used with the medical provider's inventory control software platform. In some embodiments, machine readable indicium 122 can be scanned to produce an item list of one or more medical instruments within a medical container (e.g., medical container 140). Each medical instrument on the list can then be scanned, and their working conditions can be confirmed by a surgeon, nurse, technician, etc.

In some scannable embodiments, machine readable indicium 122 can include a one-dimensional (not shown) or two-dimensional barcode (as shown in FIGS. 1-3 and 8). A one-dimensional barcode can be a Code 39 code, a VIN barcode, a Code 93 code, a Code 128 code, a GS1-128 code, or any other suitable one-dimensional barcode. A two-dimensional barcode can be a datamatrix, a QR code, a PDF417 code, a MicroPDF417 code, a MaxiCode, an Aztec code, or any other suitable two-dimensional barcode.

In some embodiments, when used with the medical provider's inventory control software platform, machine readable indicium 122 can be used to generate usage reports to give surgical teams, sterile processing processionals, management executives, etc. insight into their surgical instrumental repair and replacement programs. For example, by scanning the individual medical instruments in a medical container (e.g., medical container 140), redundancy of no longer in-use medical instruments can be eliminated and each medical instrument's repair history can be accessed.

In some embodiments, if a medical instrument in medical container 140 is damaged and/or not functioning properly, then the medical instrument can be tagged for inspection and/or repair. For example, such information can be recorded via the UDI of the medical instrument in medical container 140, and the medical instrument can be replaced with a similar instrument having its own UDI. In some embodiments, medical instruments which are sent for repair can be documented and tracked for repair frequency and costs of inspection and/or repair, and evaluated for their efficiencies such as downtime, lifespan etc. and resulting repair and replacement budgets. For example, this information can be provided in real time and accessible by management controls via reports. A medical instrument's end-of-life and functional working period can also be assessed from the information indicium compared to each manufacturer's instructions for use, providing insights into wasted expenditure and purchasing history.

In some embodiments, once each medical instrument is scanned, the medical container can be deemed ready for surgery.

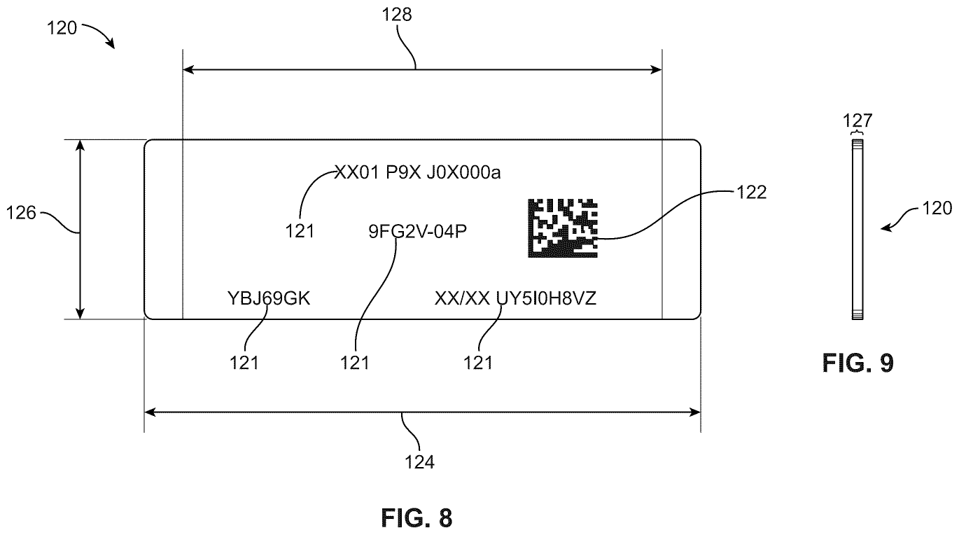

Referring to FIG. 8, information plate 120 can have a length 124 (e.g., measured from left end of information plate 120 to the right end) between about 10 mm and about 150 mm, for example, between about 30 mm and about 130 mm, between about 50 mm and about 110 mm, between about 60 mm and about 100 mm, or about 92 mm. Information plate 120 can also have a height 126 (e.g., measured from one side of information plate 120 to the other) between about 10 mm to about 150 mm, for example, between about 15 mm and about 145 mm, between about 20 mm and about 140 mm, between about 25 mm and about 135 mm, or about 30 mm. Referring to FIG. 9, information plate 120 can have a thickness 127 between about 0.1 mm and about 10 mm, for example, between about 0.5 mm and about 9.6 mm, between about 0.9 mm and about 9.2 mm, between about 1.3 mm and about 8.8 mm, or about 1.5 mm.

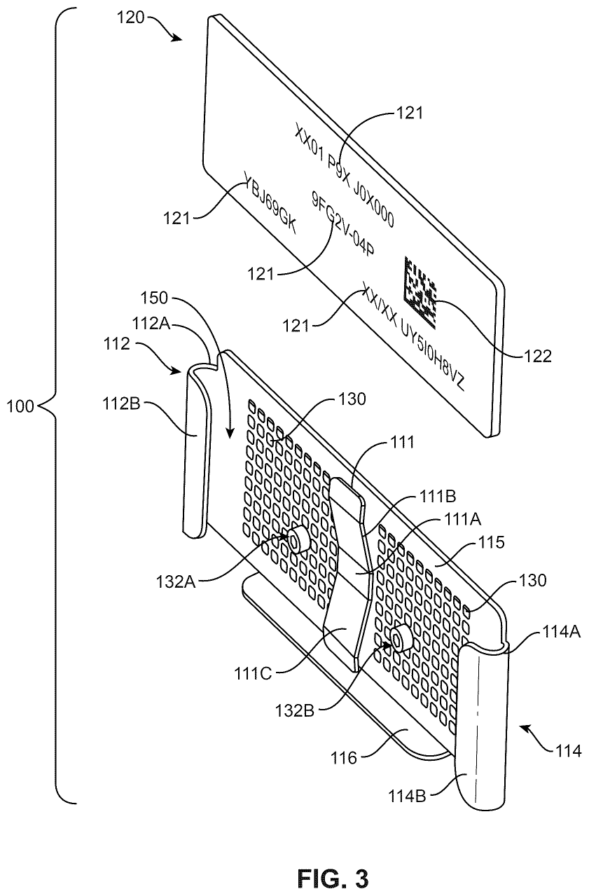

Plate holder 110 couples information plate 120 to body 141 of medical container 140. Plate holder 110 can be made of a variety of materials (e.g., stainless steel, aluminum, titanium, plastics, PEEK, etc.). In some embodiments, plate holder 110 can define a holder recess 150 configured (sized and shaped) to receive at least a portion of information plate 120. In some embodiments, plate holder 110 can include a wall surface 115, a first tab 116, a second tab 112, and a third tab 114. In some embodiments, wall surface 115, first tab 116, second tab 112, and third tab 114 can collectively define, at least in part, holder recess 150. Wall surface 115 can be substantially planar. First tab 116 extends from a first side (the bottom side as shown in FIG. 3) of wall surface 115. Second tab 112 extends from a second side (the left side as shown in FIG. 3) of wall surface 115. Third tab 114 extends from a third side (the right side as shown in FIG. 3) of wall surface 115 opposite the second side.

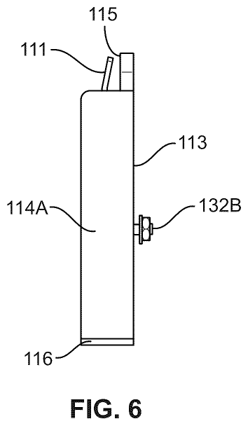

First tab 116 is configured to stop the vertical movement (downward movement in FIG. 3) of information plate 120 when positioned within holder recess 150. As shown in FIG. 6, first tab 116 can be a substantially planar flange extending perpendicular from wall surface 115.

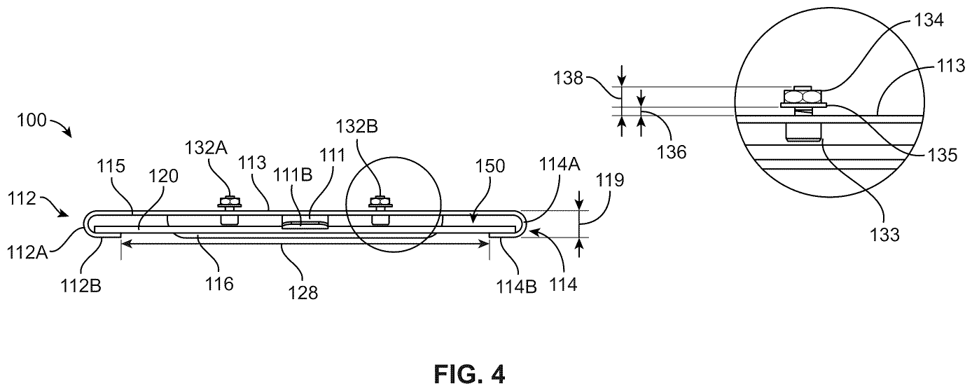

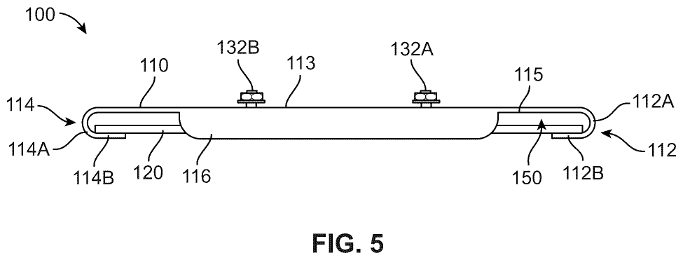

Second and third tabs 112 and 114 can be configured to substantially prevent horizontal movement of information plate 120 away from body 141 when information plate 120 is received within holder recess 150. Second tab 112 and third tab 114 can each include a proximal portion 112A, 114A extending away from wall surface 115, and a distal portion 112B, 114B extending from proximal portions 112A, 114A and spaced apart from and overlapping with a portion of wall surface 115, thereby preventing information plate 120 from moving beyond distal portions 112B and 114B and away from body 141. In some embodiments, distal portions 112B and 114B can be substantially parallel to wall surface 115. In some embodiments, as shown in FIGS. 4 and 5, tabs 112 and 114 can have a substantially U-shape when viewed from above. In some embodiments (not shown), proximal portions 112A, 114A are planar, and distal portion 112B, 114B are planar and substantially perpendicular to proximal portion 112A, 114A.

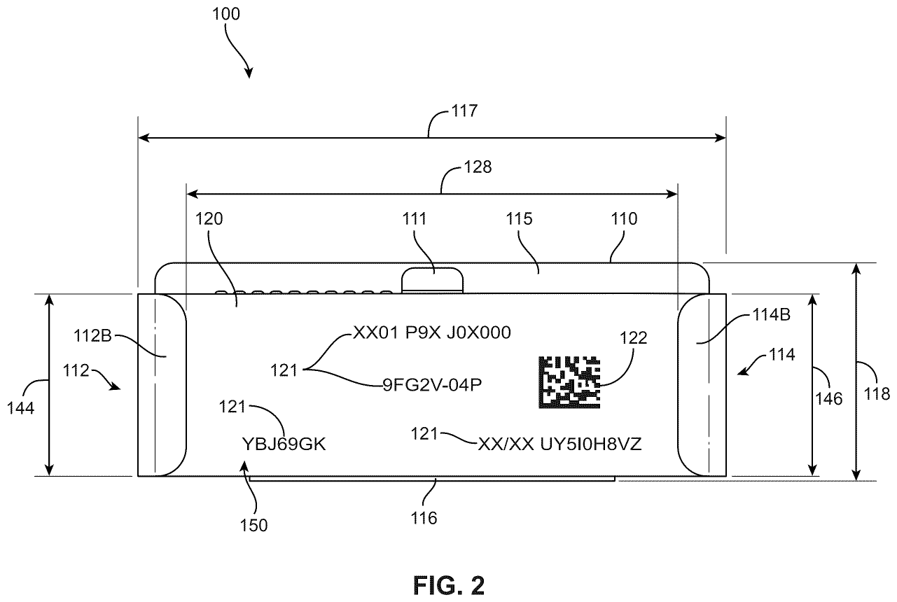

Referring to FIG. 2, second and third tabs 112 and 114 can have heights 144, 146, respectively (measured from the bottom edge of tabs 112, 114 to the top edge of tabs 112, 114 as shown in FIG. 2). In some embodiments, heights 144, 146 are substantially equal to (e.g., as shown in FIG. 2) or greater than height 126 of information plate 120. In other embodiments, heights 144, 146 are less than height 126.

In some embodiments, when plate holder 110 and information plate 120 are coupled (for example, when information plate 120 is fully received in holder recess 150), an uncovered portion of information plate 120 can have a length 128 (e.g., measured from the inner distal edge of distal portion 112B of second tab 112 to the inner distal edge of distal portion 114B of third tab 114) between about 10 mm and about 150 mm, for example, between about 30 mm and about 130 mm, between about 50 mm and about 110 mm, between about 70 mm and about 90 mm, or about 79.8 mm. Length 128 corresponds to the area on information plate 120 that is visible to a user when information plate 120 is coupled with plate holder 110.

Referring to FIG. 2, plate holder 110 can have a length 117 (e.g., measured from one end of second tab 112 to one end of third tab 114) between about 10 mm and about 180 mm, for example, between about 30 mm and about 160 mm, between about 50 mm and about 140 mm, between about 70 mm and about 120 mm, or about 97 mm. Plate holder 110 can also have a height 118 (e.g., measured from one side of wall surface 115 to the other) between about 10 mm and about 120 mm, for example, between about 15 mm and about 115 mm, between about 20 mm and about 110 mm, between about 25 mm and about 105 mm, or about 35.8 mm. Referring to FIG. 4, plate holder 110 can have a width 119 (e.g., measured from the rear side of wall surface 115 to front side of distal portion 112B, 114B and front edge of tab 116) between about 0.1 mm and about 15 mm, for example, between about 1.5 mm and about 13.6 mm, between about 2.9 mm and about 12.2 mm, between about 4.3 mm and about 10.8 mm, or about 5.6 mm.

In some embodiments, as shown for example in FIG. 3, plate holder 110 can include a retention mechanism 111 configured to couple information plate 120 to plate holder 110. For example, retention mechanism 111 can be configured to retain information plate 120 within holder recess 150 of plate holder 110. In some embodiments, retention mechanism 111 can be disposed in holder recess 150. In some embodiments, retention mechanism 111 can be a spring, a latch, etc. In some embodiments, retention mechanism 111 can press information plate 120 against at least one of second tab 112 and third tab 114, thereby creating a frictional force with tabs 112 and 114 that retains information plate 120 within holder recess 150.

In some embodiments, the spring of retention mechanism 111 can include a hub 111A coupled to wall surface 115, a first spring arm 111B extending from a first side (e.g., a top side) of hub 111A, and a second spring arm 111C extending from a second side (e.g., a bottom side) of hub 111A opposite the first side of hub 111A. Spring arms 111B and 111C contact information plate 120.

In some embodiments, first spring arm 111B can also be configured to function as a latch. For example, as shown in FIG. 4, first spring arm 111B is configured to overlap at least a portion of information plate 120 when viewed from above. This overlap can prevent information plate 120 from being accidently removed from holder recess 150 defined by plate holder 110.

In some embodiments, plate holder 110 can be coupled to medical container 140 using one or more fasteners 132A, 132B, such as a clip, nut and bolt, screw, nail, rivet, etc. For example, in some embodiments, plate holder 110 can define a plurality of holder openings 130. In some embodiments, plurality of holder openings 130 can be circles, squares, diamonds, or any combination thereof. At least a portion of the plurality of holder openings 130 can be spatially aligned to match at least a portion of the plurality of container openings 142 of medical container 140. In some embodiments, at least one fastener, for example, two fasteners 132A, 132B, can couple plate holder 110 to medical container 140. In some embodiments, each of the at least one fastener 132A, 132B can be configured to extend through one of the plurality of holder openings 130 and one of the plurality of container openings 142 to couple plate holder 110 to medical container 140. For example, in some embodiments, each of the at least one fastener 132A, 132B can be a bolt 133 and, a corresponding nut 134 and a washer 135. The shank of bolt 133 can extend through one of the plurality of holder openings 130 and one of the plurality of container openings 142, and nut 134 and washer 135 can screw unto the distal end of the shank, thereby securing plate holder 110 to medical container 140. In some embodiments, bolt 133 can be an M3×4 Allen bolt, and nut 134 can be an M2 nut.

In some embodiments, bolt 133 can have a length such that there is a gap 136 between (i) nut 134 and washer 135 and (ii) a back surface 113 of plate holder 110. Gap 136 corresponds to the thickness of the wall of body 141 defining the plurality of container openings 142. In some embodiments, as shown for example in FIG. 4, bolt 133 can have a length 138 (e.g., measured from back surface 113 of plate holder 110 into medical container 140) between about 0.1 mm and 12 mm, for example, between about 0.8 mm and 11.3 mm, between about 1.5 mm and 10.6 mm, between about 2.2 mm and 9.9 mm, or about 3.2 mm. In some embodiments, as shown for example in FIG. 4, gap 136 can have a length (e.g., measured from back surface 113 to one side of washer 135) between about 0.1 mm and 10 mm, for example, between about 0.5 mm and 9.6 mm, between about 0.9 mm and 9.2 mm, between about 1.3 mm and 8.8 mm, or about 1.5 mm.

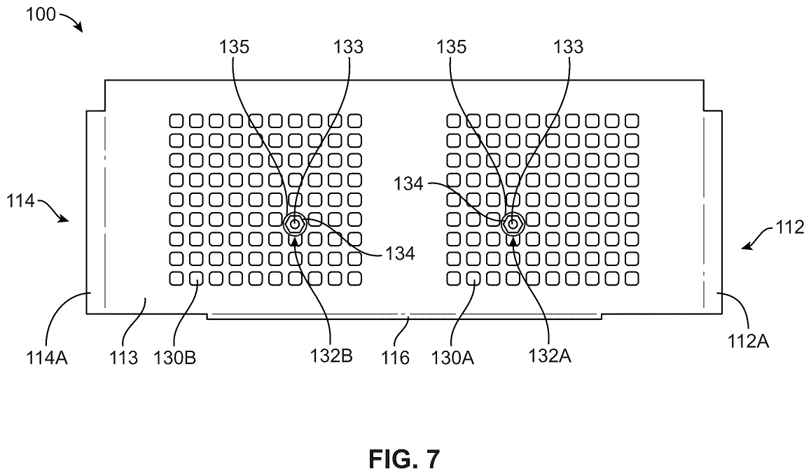

In some embodiments, plurality of holder openings 130 can include at least one grid of openings 131. In some embodiments, as shown for example in FIG. 7, plurality of holder openings 130 can include a first grid of openings 131A and a second grid of openings 131B. In some embodiments, at least one fastener 132A, 132B can include (i) a first fastener configured to extend through one of the first grid of openings 131A and one of the plurality of container openings 142 and (ii) a second fastener configured to extend through one of the second grid of openings 131B and another of the plurality of container openings 142.

In some embodiments, plate holder 110 is configured to be coupled to at least two differently configured medical device holders. For example, the plurality of holder openings 130 can be sized and spatially aligned to match both (1) at least a portion of a plurality of container openings 142 of a first type of medical container 140 and (2) at least a portion of a plurality of container openings 142 of a second type of medical container 140. The plurality of container openings 142 of the second type of medical container 140 are spaced or sized differently than the plurality of container openings 142 of the first type of medical container 140. In such embodiments, fasteners 132A can interface with (1) the plurality of holder openings 130 of plate holder 110 and (2) (i) the container openings 142 of the first type of medical container and (ii) the container openings 142 of the second type of medical container.

The foregoing description of the specific embodiments described with reference to the figures will so fully reveal the general nature of the disclosure that others can, by applying knowledge within the skill of the art, readily modify or adapt for various applications such specific embodiments, without undue experimentation, without departing from the general concept of this disclosure.

While various embodiments of the disclosure have been described above, they have been presented by way of example only, and not limitation. It should be apparent that adaptations and modifications are intended to be within the meaning and range of equivalents of the disclosed embodiments, based on the teaching and guidance presented herein. It therefore will be apparent to one skilled in the art that various changes in form and detail can be made to the embodiments disclosed herein without departing from the spirit and scope of this disclosure. The elements of the embodiments presented above are not necessarily mutually exclusive, but can be interchanged to meet various needs as would be appreciated by one of skill in the art.

It is understood that the phraseology or terminology used herein is for the purpose of description and not of limitation. The breadth and scope of this disclosure should not be limited by any of the above-described exemplary embodiments but should be defined only in accordance with the following claims and their equivalents.

It is appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

What is claimed is:

1. An information indicator assembly for medical containers defining a plurality of container openings, the assembly comprising:
an information plate comprising a machine readable indicium;
a plate holder defining a holder recess configured to receive the information plate, the plate holder further defining a plurality of holder openings that are spatially aligned to match the plurality of container openings of a first medical container; and
at least one fastener configured to extend through one of the plurality of holder openings and one of the plurality of container openings to thereby couple the plate holder to the medical container.

2. The information indicator assembly of claim 1, wherein the plurality of holder openings are spatially aligned to also match the plurality of container openings of a second medical container, the alignment of the plurality of container openings of the second medical container being different from the alignment of the plurality of container openings of the first medical container.

3. The information indicator assembly of claim 1, wherein the machine readable indicium comprises a one- or two-dimensional barcode.

4. The information indicator assembly of claim 1, wherein the machine readable indicium comprises a two-dimensional barcode.

5. The information indicator assembly of claim 4, wherein the two-dimensional barcode comprises a datamatrix or a QR code.

6. The information indicator assembly of claim 1, wherein the machine readable indicium comprises information that identifies the first medical container.

7. The information indicator assembly of claim 6, wherein the information that identifies the first medical container includes a unique device identifier.

8. The information indicator assembly of claim 6, wherein the information that identifies the first medical container indicates at least one of (a) one or more medical instruments contained in the first medical container and (b) the sterilization status of the one or more medical instruments contained in the first medical container.

9. The information indicator assembly of claim 1, wherein the plurality of holder openings comprises a grid of openings.

10. The information indicator assembly of claim 1, wherein the at least one fastener comprises a bolt and a nut.

11. The information indicator assembly of claim 10, wherein the bolt has a length such that there is a gap between the nut and a back surface of the plate holder.

12. The information indicator assembly of claim 1, wherein the plurality of holder openings comprise a first grid of openings and a second grid of openings; and wherein the at least one fastener comprises (i) a first fastener configured to extend through one of the first grid of openings and one of the plurality of container openings and (ii) a second fastener configured to extend through one of the second grid of openings and another of the plurality of container openings.

13. The information indicator assembly of claim 1, wherein the plate holder further comprises a retention mechanism disposed in the holder recess and configured to retain the information plate within the holder recess thereby coupling the information plate to the plate holder.

14. The information indicator assembly of claim 13, wherein the retention mechanism is a spring that presses the information plate against a wall surface of the plate holder.

15. The information indicator assembly of claim 1, wherein the medical container is a medical tray.

16. The information indicator assembly of claim 1, wherein the information plate further comprises a human-readable indicium.

17. A medical container for holding one or more medical instruments comprising:
a body defining an instrument cavity for containing the one or more medical instruments;
an information plate comprising a machine readable indicium; and
a plate holder comprising:
a wall surface,
a first tab extending from a first side of the wall surface,
a second tab extending from a second side of the wall surface,
a third tab extending from a third side of the wall surface opposite the second side, wherein the wall surface, the first tab, the second tab, and the third tab collectively define a holder recess configured to receive the information plate, and
a retention mechanism in the holder recess configured to couple the information plate to the plate holder.

18. The medical container of claim 17, wherein the retention mechanism is a spring that presses the information plate against at least one of the second tab and the third tab.

19. The medical container of claim 18, wherein the spring comprises a hub coupled to the wall surface, a first spring tab extending from a first side of the hub, and a second spring tab extending from a second side of the hub opposite the first side of the hub.

20. The medical container of claim 17, wherein the second and third tabs each comprise:
a proximal portion extending away from the wall surface; and
a distal portion extending from the proximal portion and spaced apart from and overlapping with a portion of the wall surface.

21. The medical container of claim 17, wherein the machine readable indicium comprises a one- or two-dimensional barcode.

22. The medical container of claim 17, wherein the machine readable indicium comprises a two-dimensional barcode, and wherein the two-dimensional barcode comprises a datamatrix or a QR code.

23. The medical container of claim 17, wherein the machine readable indicium comprises a unique device identifier.

24. The medical container of claim 17, wherein the plate holder is a separable component fastened to the body of the medical container.

25. The medical container of claim 17, wherein the plate holder is integral with the body of the medical container.

26. The medical container of claim 17, wherein the medical container is a medical tray.

27. The medical container of claim 17, wherein the information plate further comprises a human-readable indicium.
